(19) United States
(12) Patent Application Publication
(10) Pub. No.: US 2003/0149306 A1
(43) Pub. Date: Aug. 7, 2003

(54) Process for the preparation of polymorph of 4-(aryl)-1,2,3,4-tetrahydro-1-naphthale-namine derivative

(76) Inventors: Trinadha Rao Chitturi, Baroda (IN); Rajamannar Thennati, Baroda (IN)

Correspondence Address: Stephan A Pendorf, Pendorf & Cutliff, PO Box 20445, Tampa, FL, 33622-0445, US

(21) Appl. No.: 10/239,930

(22) PCT Filed: Mar. 27, 2001
(86) PCT No.: PCT/IN01/00049

(30) Foreign Application Priority Data
Mar. 29, 2000 (IN) 979/BOM/99

(51) Int. Cl. C07C 211/54

(52) U.S. Cl. 564/429

(57) ABSTRACT

A process for the preparation of a polymorph of 4-(aryl)-1,2,3,4-tetrahydro-1-naphthalenamine derivative, a hydrochloride salt of (1S,4S) N-methyl-4-(3,4-dichlorophenyl)-1,2,3,4-tetrahydro-1-naphthalenamine, comprising adding hydrochloride salt of (1S,4S) N-methyl-4-(3,4-dichlorophenyl)-1,2,3,4-tetrahydro-1-naphthalenamine to an alkanol-water solvent system, heating to dissolve and cooling the solution to allow crystallization to occur so as to obtain Form V.

### FIELD OF THE INVENTION

This invention relates to a process for the preparation of a polymorph of hydrochloride salt of (1S,4S) N-methyl-4-(3,4-dichlorophenyl)-1,2,3,4-tetrahydro-1-naphthalenamine. a compound of formula 1. The hydrochloride salt of (1S,4S) N-methyl-4-(3, 4-dichlorophenyl)-1,2,3,4-tetrahydro-1-naphthalenamine is useful in the treatments of depression, obsessive-compulsive disorder and panic disorder.

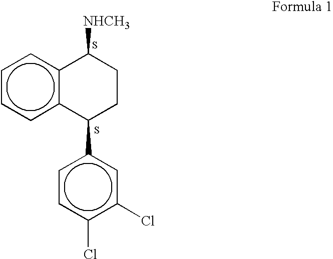

### BACKGROUND OF THE INVENTION

(1S,4S) N-methyl-4-(3,4-dichlorophenyl)-1,2,3,4-tetrahydro-1-naphthalenamine and a process for making it is disclosed in U.S. Pat. No. 4,536,518 (Welch, Jr. et al. issued Aug 20, 1985—Indian Reference not available) assigned to Pfizer, Inc.

The hydrochloride salt of (1S,4S) N-methyl-4-(3,4-dichlorophenyl)-1,2,3, 4-tetrahydro-1-naphthalenamine is known to exist in several different crystalline forms. U.S. Pat. No. 5,248,699 (Sysko et al. issued Sep. 28, 1993—Indian Reference not available) assigned to Pfizer Inc. discloses five polymorphic forms, namely Form I, Form II, Form III, Form IV and Form V. The crystal densities of the crystalline forms were 1.354, 1.314, 1.313, 1.349, and 1.308 for Form I, Form II, Form III, Form IV and Form V; respectively. The patent reports Form I which has the highest crystal density to be the most thermodynamically stable form. It is suggested that this makes Form I the most suitable crystal form for formulation, however, it has been reported by others—U. S. Pat. No. 5,734,083 (Indian Reference not available) that Form I dissolves too slowly to provide the desired bioavailability from a pharmaceutical formulation.

The processes for preparation of polymorphic forms I to V of the hydrochloride salt of (1S,4S) N-methyl-4-(3,4-dichlorophenyl)-1,2,3,4-tetrahydro-1-naphthalenamine are disclosed in U.S. Pat. No. 5,248,699 (Indian Reference not available). Form I is prepared by crystallization over a period of about 3 hours in an acidic solution using solvent such as isopropanol, hexane, ethyl acetate, acetone, methyl isobutyl ketone and glacial acetic acid at crystallization temperature from about 20° C. to about 100° C. Forms II and IV may be formed by rapid crystallization from an organic solvent. Form III is produced by heating Forms I, II or IV to above about 180° C. It is reported that Form V may be prepared by sublimation of the hydrochloride salt of (1S, 4S) N-methyl-4-(3,4 -dichlorophenyl)-1,2,3,4-tetrahydro-1-naphthalenamine at a reduced pressure at a temperature from about 180° C. to about 190° C.

U.S. Pat. No. 5,734,083 (Wilson et al, issued Mar. 31, 1998-Indian Reference not available) assigned to Torcan Chemical Ltd., (Indian Reference not available) discloses a novel polymorph T₁, which is reported to have an acceptable stability and a much-improved bioavailability as compared to Form I. This polymorph T₁ is obtained by treating a slurry or solution of free base, (1S, 4S) N-methyl-4-(3, 4-dichlorophenyl)-1,2,3,4-tetrahydro-1-naphthalenamine in an organic solvent such as toluene with a polar solvent such as ethyl acetate, diethyl ether or mixtures thereof, so as to form a solution of the free base in the polar solvent; and acidifying the mixture by the addition of a solution of hydrogen chloride (1-10% w/w) in an organic solvent such as ethyl acetate.

Polymorph Form V of (1S, 4S) N-methyl-4-(3,4-dichlorophenyl)-1,2,3, 4-tetrahydro-1-naphthalenamine hydrochloride is reportedly prepared by process of sublimation under reduced pressure. The objective of the present invention is to prepare polymorphic Form V of the hydrochloride salt of (1S, 4S) N-methyl-4-(3,4-dichlorophenyl)-1,2,3, 4-tetrahydro-1-naphthalenamine by a simple method.

Although Form I is the most stable crystalline form, we have found that Form V prepared by the process of the present invention is stable under a variety of conditions that one may encounter during processing and storage of pharmaceutical formulations. For example, it was stable for 3 months at 40° C. and 75% relative humidity or when subjected to grinding in a pestle and mortar or when heated upto 140° C. for 3 hours.

### OBJECTIVE OF THE INVENTION

The objective of the present invention is to develop a simple method to prepare Form V of the hydrochloride salt of (1S,4S) N-methyl-4-(3,4-dichlorophenyl)-1,2,3, 4-tetrahydro-1-naphthalenamine.

We have found that Form V of the hydrochloride salt of (1S,4S) N-methyl-4-(3, 4-dichlorophenyl)-1,2,3,4-tetrahydro-1-naphthalenamine may be conveniently prepared by adding the hydrochloride salt of (1S,4S) N-methyl-4-(3,4-dichlorophenyl)-1,2,3, 4-tetrahydro-1-naphthalenamine to an alkanol-water solvent system; heating to dissolve; and cooling the solution to allow crystallization to occur so as to obtain Form V.

### SUMMARY OF THE PRESENT INVENTION

Thus the present invention relates to a process for the preparation of a polymorph of hydrochloride salt of (1S,4S) N-methyl-4-(3,4-dichlorophenyl)-1,2,3, 4-tetrahydro-1-naphthalenamine, comprising adding the hydrochloride salt of (1S,4S) N-methyl-4-(3,4-dichlorophenyl)-1,2,3,4-tetrahydro-1-naphthalenamine to an alkanol-water solvent system, heating to dissolve and cooling the solution to allow crystallization to occur.

### BRIEF DESCRIPTION OF THE DRAWING

FIG. 1 is a characteristic Infrared spectrum (KBr) of Form V of hydrochloride salt of (1S,4S)-N-methyl-4-(3,4-dichlorophenyl)-1,2,3,4-tetrahydro-1-naphthalenamine prepared by the procedure described in Example 1.

### DESCRIPTION OF THE INVENTION

The present invention relates to a process for the preparation of a polymorph of hydrochloride salt of (1S,4S) N-methyl-4-(3,4-dichlorophenyl)-1,2,3, 4-tetrahydro-1-naphthalenamine, comprising adding the hydrochloride salt of (1S,4S) N-methyl-4-(3,4-dichlorophenyl)-1,2,3,4-tetrahydro-1 -naphthalenamine to an alkanol-water solvent system, heating to dissolve and cooling the solution to allow crystallization to occur so as to obtain Form V.

The volume of alkanol-water solvent system that may be used may range from about 6 to 15 parts per unit weight of the hydrochloride salt of (1S,4S) N-methyl-4-(3, 4-dichlorophenyl)-1,2,3,4-tetrahydro-1-naphthalenamine, preferably 8 to 10 parts per unit weight of the hydrochloride salt of (1S,4S) N-methyl-4-(3,4-dichlorophenyl)-1,2,3, 4-tetrahydro-1-naphthalenamine and the most preferred being 9 parts per unit weight of the hydrochloride salt of (1S,4S) N-methyl-4-(3,4-dichlorophenyl)-1,2,3,4-tetrahydro-1-naphthalenamine. The hydrochloride salt of (1S,4S) N-methyl4-(3,4-dichlorophenyl)-1,2,3, 4-tetrahydro-1-naphthalenamine may be suspended in alkanol-water solvent system or hydrochloride salt of (1S,4S) N-methyl-4-(3,4-dichlorophenyl)-1,2,3, 4-tetrahydro-1-naphthalenamine may be added to alkanol-water solvent system and heated for its dissolution. Hence, the sequence of addition is inconsequential.

According to an embodiment of the process of the present invention the alkanol-water system may have a relative proportion ranging from 5:1 to 20:1 parts by volume, preferably 5:1 to 10:1 parts by volume and most preferably 8:1 parts by volume.

According to a preferred embodiment of the process of the present invention the alkanol is selected from C₁ to C₄ alkanol, more preferably methanol or 2-propanol.

According to the process of the present invention the most preferred alkanol is 2-propanol.

According to one embodiment of the process of the present invention a polyol, selected from glycerol, mannitol, sorbitol, inositol, xylitol, 1,3-butanediol, 1,2-propanediol and the like, is added to the alkanol-water solvent system. Generally 0 to 25 mole % of the polyol with respect to hydrochloride salt of (1S,4S) N-methyl-4-(3,4-dichlorophenyl)-1,2,3, 4-tetrahydro-1-naphthalenamine, is added to the solvent system and preferably 10 mole %.

The polyol may be incorporated into the alkanol-water system before, during or after the stage of dissolution of hydrochloride salt of (1S,4S) N-methyl-4-(3 ,4-dichlorophenyl)-1,2,3, 4-tetrahydro-1-naphthalenamine with heating. The polyol may be incorporated preferably as in Example 1 i.e. dissolved in water and then alkanol added because the dissolution of certain polyol like mannitol is slow in alkanol-water mixture. However, the sequence of addition of the polyol should not affect the formation of form V, since the crystallization is occurring from a clear solution.

According to the process of the present invention the dissolution of hydrochloride salt of (1S,4S) N-methyl-4-(3,4-dichlorophenyl)-1,2,3,4-tetrahydro-1-naphthalenamine may be achieved by heating the solution. Generally the solution is heated to a temperature greater than about 40° C.

According to the preferred embodiment of the process of the present invention the dissolution of hydrochloride salt of (1S,4S) N-methyl-4-(3,4-dichlorophenyl)-1,2,3, 4-tetrahydro-1-naphthalenamine may be achieved by heating the solution to reflux temperature.

According to the process of the present invention the solution may be cooled to below room temperature to allow crystallization to occur.

According to the preferred embodiment of the process of the present invention the solution is cooled to 20-300° C. to allow crystallization to occur.

According to yet another embodiment of the process of the present invention the solution is allowed to cool for a period of 2 to 8 hours to allow crystallization to occur.

According to the most preferred embodiment of the process of the present invention the solution is cooled to 20-30° C. over a period of 2 to 8 hours to allow crystallization to occur.

According to the process of the present invention the product may be dried using any conventional drying techniques which may be suitable for the product such as fluidized bed drying, tray drying, rotary drying, drying at reduced pressures, freeze drying or spray drying. Driers that have agitational means are preferred.

### EXAMPLES

The invention is further illustrated but not restricted by the description in the following examples.

### Example 1

40.0 g (0.2189 moles) of mannitol is dissolved in 1.05 L water and to this solution is added 6.0 L of 2-propanol, followed by hydrochloride salt of (1S,4S) N-methyl-4-(3,4-dichlorophenyl)-1,2,3,4-tetrahydro-1-naphthalenamine, 750.0 g (2.189 moles). The suspension is heated to reflux temperature until a clear solution results (1-1.5hr). The solution is then gradually cooled to 25-30° C. (6 hr) to crystallize and the mixture further stirred at 25-30° C. for 3 hr. The product is filtered, washed with 400.0 ml 2-propanol and dried in air oven at 60° C. to constant weight.

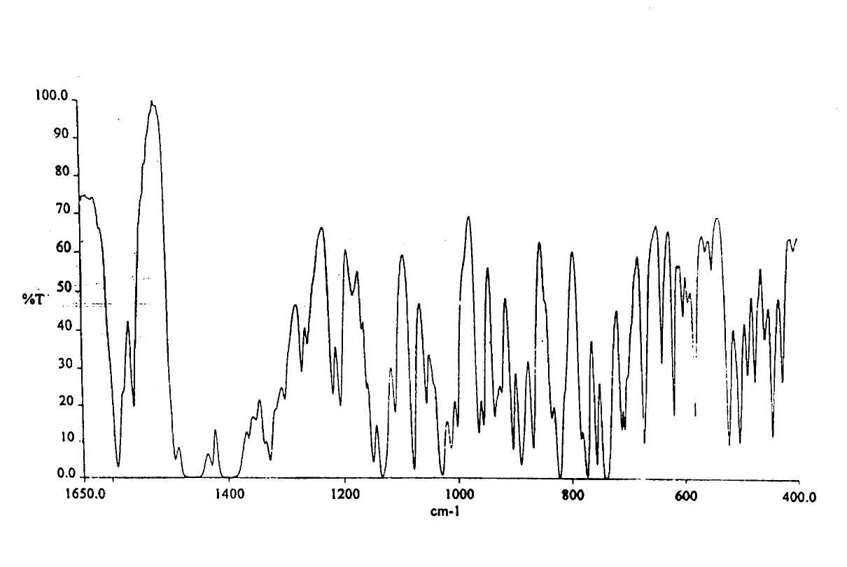

The Infrared spectrum (KBr) showed that product was Form V of the hydrochloride salt of (1S,4S) N-methyl-4-(3,4-dichlorophenyl)-1,2,3,4-tetrahydro-1-naphthalenamine. The infrared spectrum is given in FIG. 1.

No change in Infrared spectrum (KBr) and X-ray diffractogram was observed after 3 months at 40° C. and 75% relative humidity or when subjected to grinding in a pestle and mortar or when heated upto 140° C. for 3 hours.

### Example 2

5 g (14.59) of hydrochloride salt of (1S,4S) N-methyl-4-(3,4-dichlorophenyl)-1,2,3, 4-tetrahydro-1-naphthalenamine, is suspended in 40 ml of 2-propanol, 5 ml water is added and the mixture refluxed until a clear solution resulted (10-15 min). The resulting solution is gradually cooled to 25-30° C., and then further stirred at this temperature for 3 hr. The product is filtered and dried in air oven at 60° C. to constant weight.

This experiment was repeated using 2-propanol quantities ranging from 5 to 20 parts volume with respect to water. In all the cases same polymorph (form V) was obtained.

The process was repeated with 10 kg input of hydrochloride salt of (1S,4S) N-methyl-4-(3,4-dichlorophenyl)-1,2,3,4-tetrahydro-1-naphthalenamine. The Infrared spectrum (KBr) and X-ray diffractogram showed that Form V of hydrochloride salt of (1S,4S) N-methyl-4-(3,4-dichlorophenyl)-1,2,3,4-tetrahydro-1-naphthalenamine was obtained in a yield of 81.7%.

### Example 3

Method of example 1 was followed except that sorbitol was used in the place of mannitol. Form V of the hydrochloride salt of (1S,4S) N-methyl-4-(3,4-dichlorophenyl)-1,2,3, 4-tetrahydro-1-naphthalenamine was obtained.

### Example 4

The crystal form of hydrochloride salt of (1S,4S) N-methyl-4-(3,4-dichlorophenyl)-1,2,3, 4-tetrahydro-1-naphthalenamine prepared as per Examples 1 and 2 was subjected to powder x-ray diffraction analysis using copper x-ray source. Table 1 gives the x-ray diffraction data and Table 2 gives the crystal parameters for the hydrochloride salt of (1S,4S) N-methyl-4-(3,4-dichlorophenyl)-1,2,3,4-tetrahydro-1-naphthalenamine prepared as per Examples 1 and 2

TABLE 1
X-ray Diffraction Data

| Example 1 | | | Example 2 | | |
|---|---|---|---|---|---|
| Degrees 2θ | D | Intensity % | Degrees 2θ | d | Intensity % |
| 10.5 | 8.4 | 28.0 | 10.6 | 8.3 | 29.7 |
| 11.1 | 8.0 | 100.0 | 11.1 | 7.9 | 100.0 |
| 14.3 | 6.2 | 36.5 | 14.4 | 6.2 | 33.1 |
| 16.5 | 5.4 | 20.4 | 16.6 | 5.3 | 26.3 |
| 17.3 | 5.1 | 28.3 | 17.4 | 5.1 | 29.4 |
| 19.8 | 4.5 | 33.6 | 19.9 | 4.5 | 42.4 |
| 25.5 | 3.5 | 77.2 | 25.5 | 3.5 | 92.9 |
| 26.1 | 3.4 | 28.8 | 26.1 | 3.4 | 36.4 |
| 29.2 | 3.1 | 29.6 | 29.2 | 3.1 | 39.8 |

TABLE 2
Crystal Parameters

| | | Example 1 | Example 2 |
|---|---|---|---|
| | Units | Monoclinic | Monoclinic |
| a | Angstrom | 17.423 | 17.314 |
| b | Angstrom | 9.138 | 9.172 |
| c | Angstrom | 5.545 | 5.548 |
| α | in degrees | 90.00 | 90.00 |
| β | in degrees | 104.07 | 104.23 |
| γ | in degrees | 90.00 | 90.00 |
| V | Angstrom³ | 856.75 | 854.15 |

1. A process for the preparation of a polymorphy of hydrochloride salt of (1S, 4S) N-methyl-4-(3,4,-dichlorophenyl)-1,2,3,4tetrahydro-1-naphthalenamine, comprising adding the hydrochloride salt of (1S,4S) N-methyl-4-dichlorophenyl-1,2,3,4-tetrahydro-1-napthalanamino to an alkanol-water solvent system containing a polyol selected from glycerol, mannitol, sorbitol, inositol, xylitol, 1,3-butanediol and 1,2-propanediol, heating to dissolve and cooling the solution to allow crystallization to occur so as to obtain Form V.

2. A process as claimed in claim 1 wherein the alkanol is selected from C₁ to C₄ alkanol.

3. A process as claimed in claim 2 wherein the alkanol is 2-propanol.

4. A process as claimed in claim 1 wherein the solution is cooled to 20-30° C.

5. A process as claimed in claim 1 wherein the crystallization occurs over a period of about 2 to 8 hours.

6. A process as claimed in claim 1 wherein the solution is cooled to 20-30° C. over a period of about 2 to 8 hours to allow crystallization to occur.

7. A process as claimed in claim 1 wherein the hydrochloride salt of (1S,4S) N-methyl-4-(3,4,dichlorophenyl)-1,2,3,4-tetrahydro-1-napthalenamine exhibits an x-ray powder diffraction pattern having characteristic peaks expressed in degrees 28 at approximately 10.5, 11.1, 14.3, 16.5, 17.3, 19.8, 25.5, 26.1, 29.2.

8. A process as claimed in claims 1 to 6 substantially as herein described and illustrated by examples 1 to 4.
